(19) United States
(12) Patent Application Publication
(10) Pub. No.: US 2024/0144025 A1
(43) Pub. Date: May 2, 2024

(54) INFORMATION PROCESSING DEVICE, INFORMATION PROCESSING METHOD, PROGRAM

(71) Applicant: NEC Corporation, Tokyo (JP)

(72) Inventor: Kosuke NISHIHARA, Tokyo (JP)

(73) Assignee: NEC Corporation, Tokyo (JP)

(21) Appl. No.: 18/382,714

(22) Filed: Oct. 23, 2023

(30) Foreign Application Priority Data
Oct. 31, 2022 (JP) 2022-174357

(51) Int. Cl. G06N 3/094; A61B 5/11

(52) U.S. Cl. CPC G06N 3/094; A61B 5/11

(57) ABSTRACT

An information processing device includes a feature extraction means for extracting, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person; a motion data generation means for generating first motion data based on the basic feature data and the motion feature data, and generating second motion data based on the basic feature data and the person feature data; and a learning means for learning the feature extraction means and the motion data generation means based on the first motion data and the second motion data.

### INCORPORATION BY REFERENCE

The present invention is based upon and claims the benefit of priority from Japanese patent application No. 2022-174357, filed on Oct. 31, 2022, the disclosure of which is incorporated herein in its entirety by reference.

### TECHNICAL FIELD

The present disclosure relates to an information processing device, an information processing method, and a program.

### BACKGROUND ART

In promoting exercise to prevent nursing care and frailty, there is a demand to recognize effects in improving motions by the exercise in advance so as to give motivation to start and continue the exercise. Even in the medical field, there is a demand to predict a change in motions along with a change in the disease condition of a patient. As a technology related to such demands, Non-Patent Literature 1 describes conversion of a motion of a subject. Specifically, in Non-Patent Literature 1, motion images are configured of basic motions (for example, walk, kick) and motion styles (motion features (for example, neutral, old)), and Non-Patent Literature 1 describes conversion of a motion style without changing the basic motion.
Non-Patent Literature 1: Aberman, K., Weng, Y, Lischinski, D., Cohen-Or, D., & Chen, B. (2020). “Unpaired motion style transfer from video to animation.” ACM Transactions on Graphics (TOG), 39(4), 64-1.

### SUMMARY

However, in the technology of Non-Patent Literature 1, while a change in a motion of a person can be predicted by changing the motion style from the basic motion, a person has characteristics in the motion, and such characteristics cannot be reflected. Therefore, there is a problem that a change in the motion of a person cannot be predicted with high accuracy.

Therefore, an object of the present invention is to solve the problem described above, that is, a problem that a change in the motion of a person cannot be predicted with high accuracy.

An information processing device, according to one aspect of the present disclosure, is configured to include
a feature extraction means for extracting, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
a motion data generation means for generating first motion data on the basis of the basic feature data and the motion feature data, and generating second motion data on the basis of the basic feature data and the person feature data; and
a learning means for learning the feature extraction means and the motion data generation means on the basis of the first motion data and the second motion data.

Further, an information processing method, according to one aspect of the present disclosure, is configured to include
by a feature extraction means, extracting, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation means, generating first motion data on a basis of the basic feature data and the motion feature data, and generating second motion data on the basis of the basic feature data and the person feature data; and
by a learning means, learning the feature extraction means and the motion data generation means on the basis of the first motion data and the second motion data.
further, a program, according to one aspect of the present disclosure, causes a computer to execute processing to
by a feature extraction means, extract, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation means, generate first motion data on a basis of the basic feature data and the motion feature data, and generate second motion data on the basis of the basic feature data and the person feature data; and
by a learning means, learn the feature extraction means and the motion data generation means on the basis of the first motion data and the second motion data.

With the configurations described above, the present invention can predict a change in a motion of a person with high accuracy.

### BRIEF DESCRIPTION OF DRAWINGS

FIG. 1 illustrates the outline of processing performed by an information processing device of the present disclosure;

FIG. 2 illustrates the outline of processing performed by the information processing device of the present disclosure;

FIG. 3 illustrates the outline of processing performed by the information processing device of the present disclosure;

FIG. 4 is a block diagram illustrating a configuration of an information processing device according to a first example embodiment of the present disclosure;

FIG. 5 illustrates a state of processing by the information processing device disclosed in FIG. 4;

FIG. 6 illustrates a state of processing by the information processing device disclosed in FIG. 4;

FIG. 7 illustrates a state of processing by the information processing device disclosed in FIG. 4;

FIG. 8 is a flowchart illustrating an operation of the information processing device disclosed in FIG. 4;

FIG. 9 illustrates another configuration of and another state of processing by the information processing device disclosed in FIG. 4;

FIG. 10 illustrates another configuration of and another state of processing by the information processing device disclosed in FIG. 4;

FIG. 11 is a block diagram illustrating a hardware configuration of an information processing device according to a second example embodiment of the present disclosure; and

FIG. 12 is a block diagram illustrating a configuration of the information processing device according to the second example embodiment of the present disclosure.

### EXAMPLE EMBODIMENTS

### First Example Embodiment

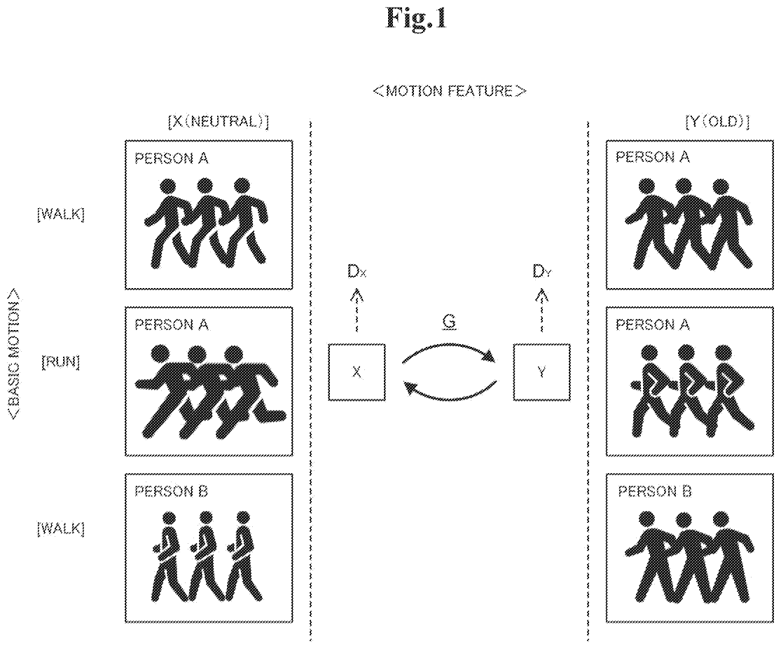

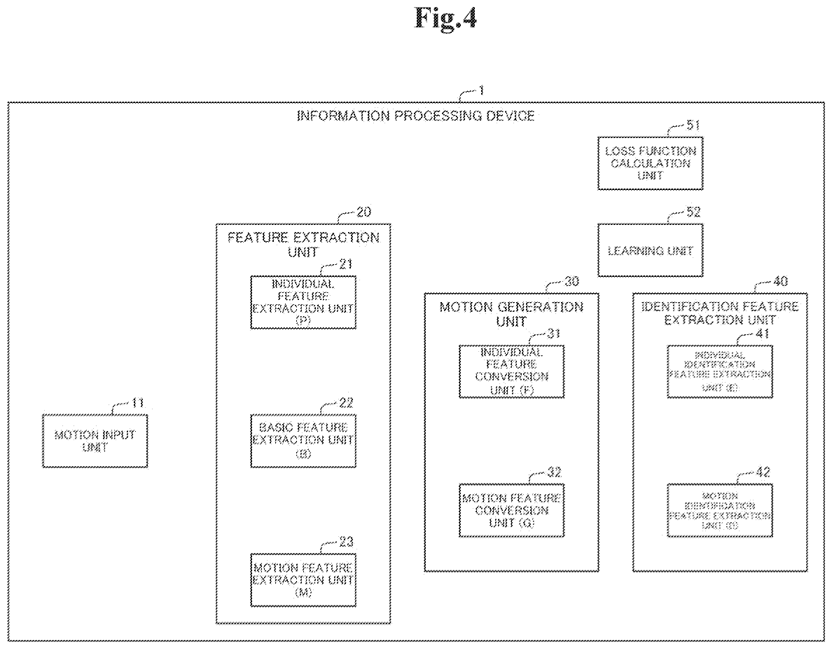

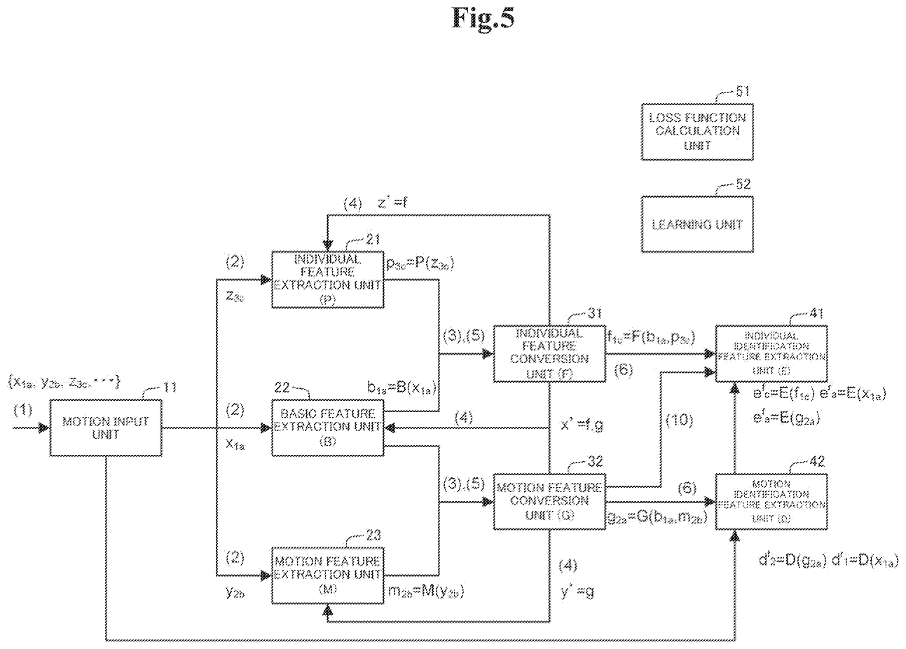

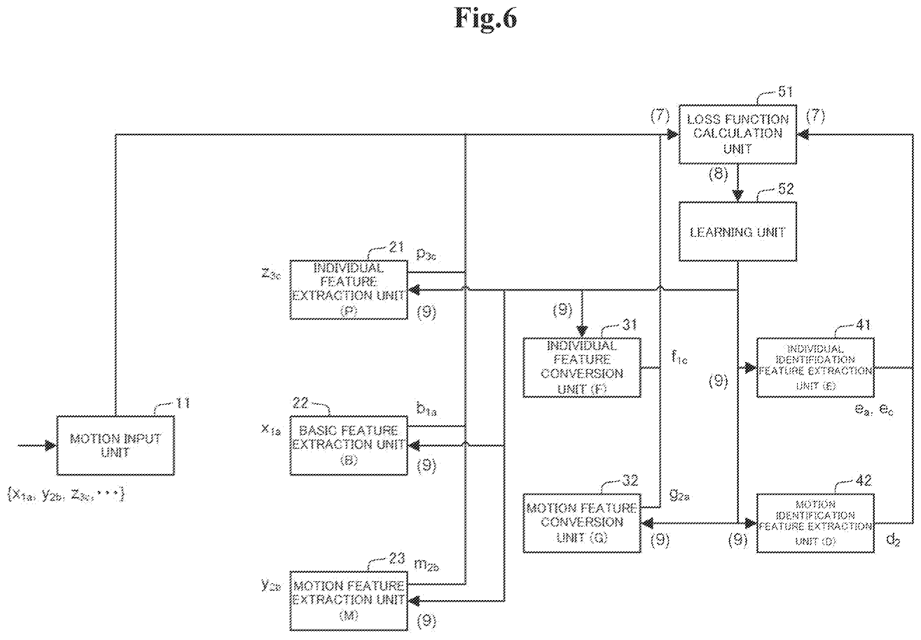

A first example embodiment of the present invention will be described with reference to FIGS. 1 to 10. FIGS. 1 to 3 illustrate the outline of processing performed by an information processing device. FIG. 4 is a diagram for explaining a configuration of the information processing device, and FIGS. 5 to 10 are diagrams for explaining processing operation of the information processing device.

[Outline]

First, the outline of the present disclosure will be described. An information processing device 1 of the present disclosure is used for generating motion data of a person and, for example, generating motion data to predict a motion of a person. Therefore, the information processing device 1 has a function of learning existing motion data of a person and generating a model for generating new motion data. In particular, the information processing device 1 has a function of generating a model to convert motion data of an existing person into new motion data consisting of a motion having a motion style to be predicted reflecting an individual feature of a person.

FIGS. 1 to 3 illustrate the outline of learning by the information processing device 1. First, as motions of a person, there are “basic motions”. As examples thereof, FIG. 1 illustrates motion data of “walk” and “run”. Further, as motions of a person, there are “motion styles (motion features)”. As examples thereof, FIG. 1 illustrates motion data of “neutral” and “old”. Furthermore, FIG. 1 illustrates motion data of a plurality of persons such as persons A to D.

The information processing device 1 extracts, from the motion data as described above, “basic feature data” representing the feature of each “basic motion”, “motion feature data” representing the feature of each “motion style”, and “individual feature data (person feature data)” representing the feature of each person. Then, by using the “basic feature data” and the “motion feature data”, the information processing device 1 performs learning of a model to convert motion data of a motion style into motion data of another motion style. The example of FIG. 1 illustrates the case of learning a conversion model G to convert a motion style between “neutral: X” and “old: Y” among the “motion styles”. In this case, when there is a motion data pair corresponding to “neutral: X” and “old: Y” that are two motion styles of the same person, the information processing device 1 can perform learning using the motion data pair. Meanwhile, even when there is no motion data pair, it is also possible to learn the conversion model G by performing circulation learning. Specifically, the information processing device 1 inversely converts the motion data in which the motion style “X” was converted to “Y”, that is, inversely converts the converted “Y” to “X”, and learns the conversion model G so as to allow the error between the inversely converted motion data and the original motion data to be small. Thereby, it is possible to generate the conversion model G for converting the motion style even though there is no motion data pair before and after the motion.

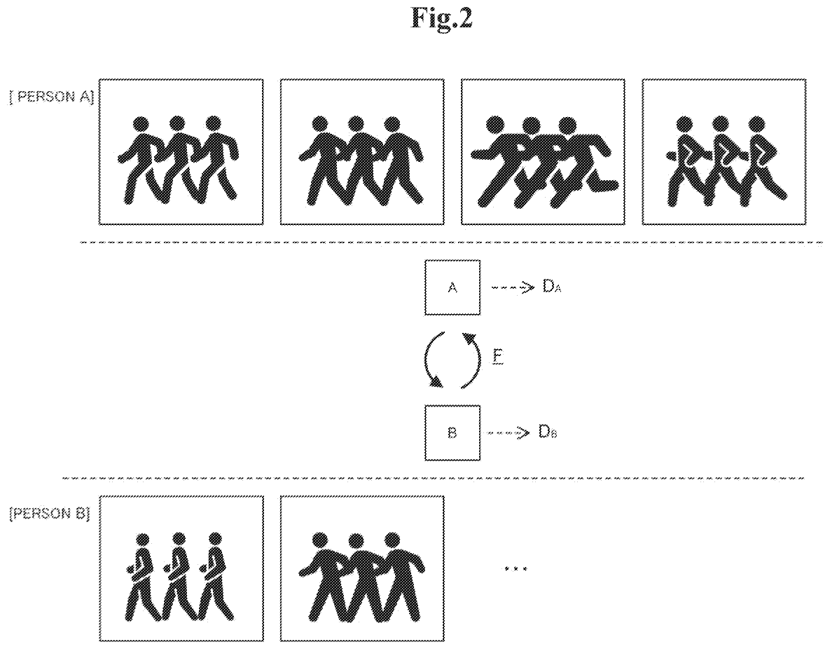

Moreover, the information processing device 1 performs learning of a model to convert “individual feature” by using the “basic feature data” and the “individual feature data”. In the example of FIG. 2, a conversion model F for converting the individual feature between the “person: A” and the “person: B” among the “individual features” is learned. In this case, when there is a motion data pair of the same basic motion and motion style between the persons, learning can be performed by using the motion data pair. Meanwhile, even when there is no motion data pair of the same basic motion and motion style between the persons, it is also possible to learn the conversion model F by performing circulation learning. Specifically, the information processing device 1 inversely converts the motion data in which the individual feature “A” was converted to “B”, that is, inversely converts the converted “B” to “A”, and learns the conversion model F so as to allow the error between the inversely converted motion data and the original motion data to be small. Even when there is no motion data pair between the persons, it is possible to generate the conversion model F.

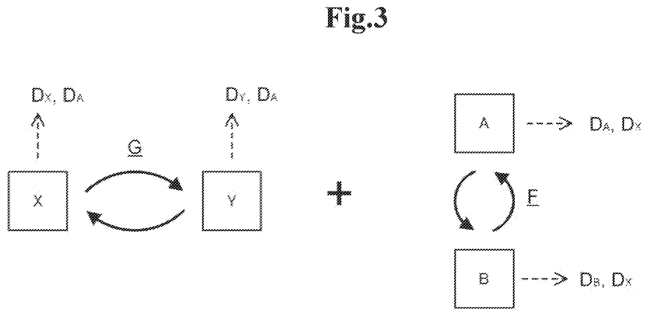

Then, by simultaneously learning the conversion model G for converting the “motion style” and the conversion model F for converting the “individual feature” that are generated as described above, the information processing device 1 can generate a model that converts the motion style while reflecting the individual feature. For example, in the example of FIG. 3, it is possible to optimize the conversion model G for converting the motion style by reflecting the feature of the person A learned in the conversion model F for converting the individual feature. Thereby, by inputting the motion data of the motion style X of the person A for example to the generated model, it is possible to generate motion data in which the motion style is converted to the motion style Y while reflecting the individual feature of the person A. Note that by inputting the motion data of the motion style X of the person A to the generated model, it is also possible to generate motion data in which the individual feature is converted to another person B, without changing the motion style X.

Generation of motion data as described above is also applicable to various motions constituting the “basic motion” and “motion style”. The “basic motion” includes “{walk, run, jump, kick, punch} and the like. The “basic motion” may also include a motion for checking the condition related to a disease, for example, a motion such as {stand/sit}. Further, the “motion style” includes {neutral, child-like, old-like, angry} and the like. The “motion style” may also include conditions related to a disease such as {sound, moderate, serious}.

By using various types of motion data as described above in the information processing device 1 of the present disclosure, it is possible to generate motion data in various situations of a person while reflecting the individual feature of the person. For example, from the motion data of the case where a person has a motion style of “moderate” condition of a disease due to lack of exercise, it is possible to generate motion data of the case where the person becomes to have a motion style of “sound” after the exercise while reflecting the individual feature of the person, and predict a motion.

[Configuration]

Next, a configuration of the information processing device 1 of the present embodiment will be described with reference to FIG. 4. Note that here describes the outline of the configuration of the information processing device 1. Specific functions of the respective constituent elements will be described in detail in the description of operation provided thereafter.

The information processing device 1 is configured of one or a plurality of information processing devices each having an arithmetic device and a storage device. As illustrated in FIG. 4, the information processing device 1 includes a motion input unit 11, a feature extraction unit 20, a motion generation unit 30, an identification feature extraction unit 40, a loss function calculation unit 51, and a learning unit 52. The feature extraction unit 20 includes an individual feature extraction unit 21, a basic feature extraction unit 22, and a motion feature extraction unit 23. The motion generation unit 30 includes an individual feature conversion unit 31 and a motion feature conversion unit 32. The identification feature extraction unit 40 includes an individual identification feature extraction unit 41 and a motion identification feature extraction unit 42. The functions of the respective units can be implemented through execution, by the arithmetic device, of a program for implementing respective functions stored in the storage device. Hereinafter, the respective constituent elements will be described in detail.

In the learning phase, the motion input unit 11 inputs prepared motion data to the basic feature extraction unit 22, the motion feature extraction unit 23, the individual feature extraction unit 21, the identification feature extraction unit 40, and the loss function calculation unit 51. In the inference phase, the motion input unit 11 inputs motion data to be converted, to the basic feature extraction unit 22 and the motion feature extraction unit 23, and inputs motion data having a motion style to be converted, to the motion feature extraction unit 23.

The basic feature extraction unit 22 (feature extraction means) extracts, from the input motion data, a basic feature vector (basic feature data) that represents the feature of a basic motion. Motion data is, for example, data in which coordinates of a joint and the rotation angle continue in a plurality of frames. The examples include one in which coordinate points of (x, y, z) and a rotation angle such as an Euler angle continue for 64 frames for each of the twenty-three joints such as a neck and knees. The basic feature extraction unit 22 consists of a “model B” by a neural network, and when motion data is input as described above, it converts the data to a basic feature vector. A basic feature vector is, for example, a vector having elements of 256 elements or 512 elements obtained by reducing the dimensions of the input data. Note that the model of a neural network is not limited particularly.

The motion feature extraction unit 23 (feature extraction means) extracts, from the input motion data, a motion feature vector (motion feature data) that represents the motion style (motion feature). The motion feature extraction unit 23 consists of a “model M” by a neural network, and when motion data is input as described above, it converts the data to a motion feature vector. Note that the model of a neural network is not limited particularly.

The individual feature extraction unit 21 (feature extraction means) extracts, from the input motion data, an individual feature vector (person feature data) that represents the feature of a person. The individual feature extraction unit 21 consists of a “model P” by a neural network, and when motion data is input as described above, it converts the data to an individual feature vector. Note that the model of a neural network is not limited particularly.

The motion generation unit 30 (motion data generation means) receives the respective feature vectors as inputs, and generates motion data by using the neural network. Specifically, the motion generation unit 30 is configured of the individual feature conversion unit 31 that generates motion data (second motion data) in which the individual feature is converted based on the individual feature vector and the basic feature vector described above, and the motion feature conversion unit 32 that generates motion data (first motion data) in which the motion style is converted based on the motion feature vector and the basic feature vector described above. The individual feature conversion unit 31 is configured of the “model F” consisting of a neural network, and the motion feature conversion unit 32 is configured of the “model G” consisting of a neural network. Note that the model of a neural network is not limited particularly. The motion generation unit 30 may input a vector in which the basic feature vector and the motion feature vector are connected to the individual feature vector, to the neural network. Alternatively, the motion generation unit 30 may input the basic feature vector to the neural network, and obtain a final output by adding or multiplying the individual feature vector or the motion feature vector to the intermediate output. The motion generation unit 30 outputs the generated motion data not only to the identifying feature extraction unit 40, but also to the basic feature extraction unit 22, the motion feature extraction unit 23, and the individual feature extraction unit 21.

The identification feature extraction unit 40 (identification feature extraction means) is configured of a person identification feature extraction unit 41 for identifying whether a motion having an individual feature is a motion generated by the motion generation unit 30 or an input motion, and a motion identification feature extraction unit 42 for identifying whether a motion having a motion style is a motion generated by the motion generation unit 30 or an input motion. The individual identification feature extraction unit 41 is configured of a “model E” consisting of a neural network, and the motion identification feature extraction unit 42 is configured of a “model D” consisting of a neural network. Note that the model of a neural network is not limited particularly. The individual identification feature extraction unit 41 and the motion identification feature extraction unit 42 convert the data input as described below into a feature vector or a scalar value, and generates and outputs an individual identification feature and a motion identification feature (identification feature value).

The loss function calculation unit 51 (learning means) calculates a difference from a correct label by using a loss function. Loss functions include an adversarial learning loss function for identifying whether or not data generated by the individual feature conversion unit 31 or the motion feature conversion unit 32 is generated data, a classification loss function for calculating a difference between the feature vector extracted by the basic feature extraction unit 22, the motion feature extraction unit 23, or the individual feature extraction unit 21 and a corresponding correct label, and an error loss function for calculating a difference between motion data cyclically generated by the individual feature conversion unit 31 or the motion feature conversion unit 32 and input data. However, loss functions are not limited to those described above, and a necessary loss function may be added.

The learning unit 52 (learning means) performs learning by using an optimization method of a neural network based on the value of a loss function, and updates the weight of the neural network constituting each of the models (P, B M, F, G, E, D). Note that the method of updating the weight of a neural network is not limited particularly.

[Operation]

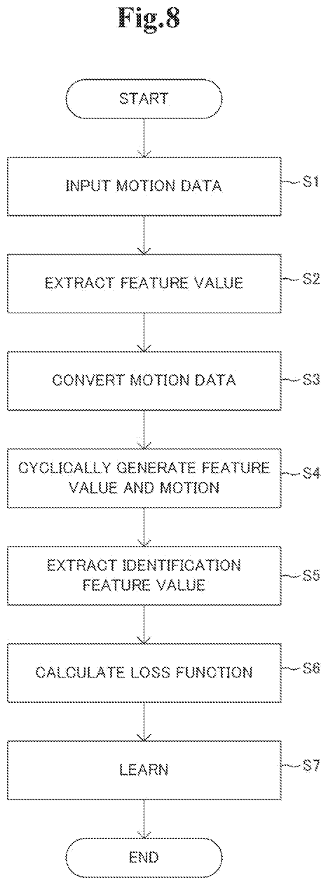

Next, operation of the information processing device 1 described above will be described with reference to FIGS. 5 to 7 and the flowchart of FIG. 8. First, a learning phase to generate a model will be described. In FIGS. 5 to 7, the operation sequence is shown by the numbers in brackets written alongside the arrows.

(1) Read Motion Data

First, the motion input unit 11 reads motion data from a dataset. Each unit of motion data has a label {basic motion, motion style, individual feature}. For example, each unit of motion data is represented as {basic motion_{motion style, individual feature}}. The basic motion is labeled with {walk, run, jump, kick, punch. stand/sit, . . . } and the like, which is represented as {x, y, z, . . . } in this example. The individual feature is labeled with {person A, person B, person C, . . . } and the like, which is represented as {a, b, c, . . . } in this example. The motion style (motion feature) may be represented as motion styles such as {neutral, child-like, old-like, angry, . . . }, or represented as motion levels such as {sound, moderate, serious, . . . }. The motion style may be different depending on the use case. In this example, it is represented as {1, 2, 3, . . . }. Then, the input motion data is collectively represented as {x₁ₐ, y_{2b}, z_{3c}, . . . } and the like. For example, {x₁ₐ} represents motion data of basic motion: x, motion style: 1, and individual feature: person A.

(2) Input Motion

The motion input unit 11 inputs motion data to the basic feature extraction unit 22, the motion feature extraction unit 23, and the individual feature extraction unit 21 (step S1). A plurality of units of motion data may be input collectively. Motion data to be input may be selected randomly, or may be selected according to a predetermined rule.

(3) Extract Feature Value

The basic feature extraction unit 22, the motion feature extraction unit 23, and the individual feature extraction unit 21 convert input motion data into feature vectors by using the models B, M, and P each consisting of a neural network (step S2). For example, the basic feature extraction unit 22 receives motion data x₁ₐ as an input and outputs a basic feature vector b₁ₐ, the motion feature extraction unit 23 receives motion data y_{2b} as an input and outputs a motion feature vector m_{2b}, and the individual feature extraction unit 21 receives data z_{3c} as an input and outputs an individual feature vector p_{3c}, respectively.

(4) Generate Motion

The individual feature conversion unit 31 generates motion data f_{1c} by converting the individual feature, based on the individual feature vector p_{3c} and the basic feature vector bi a (step S3). The motion feature conversion unit 32 generates motion data g₂ₐ by converting the motion style, based on the motion feature vector m_{2b} and the basic feature vector b₁ₐ (step S3). At that time, the individual feature conversion unit 31 and the motion feature conversion unit 32 input the basic feature vector to the models F and G each consisting of a neural network respectively, and add or multiply the individual feature vector or the motion feature vector to the intermediate output to thereby obtain a final output described above.

(5) Cyclically Generate Feature Value

The basic feature extraction unit 22 receives the motion data f_{1c} generated by the individual feature conversion unit 31 and the motion data g₂ₐ generated by the motion feature conversion unit 32 as inputs again, and outputs basic feature vectors b_{1c} and b₂ₐ, respectively. The motion feature extraction unit 23 receives the motion data x₁ₐ as an input and outputs motion feature vector m₁ₐ. The individual feature extraction unit 21 receives the motion data x₁ₐ as an input and outputs individual feature vector pia.

(6) Cyclically Generate Motion

The individual feature conversion unit 31 generates motion data f₁ₐ by converting the individual feature, based on the individual feature vector pi a and the basic feature vector bi, again. The motion feature conversion unit 32 generates motion data g₁ₐ by converting the motion style, based on the motion feature vector m₁ₐ and the basic feature vector b₂ₐ again. As described above, the motion data f_{1c} and the motion data g₂ₐ having been generated through conversion from the input data are inversely converted, and the motion data f₁ₐ and g₁ₐ that may correspond to the input data x₁ₐ are generated (step S4).

(7) Extract Identification Feature

The identification feature extraction unit 40 inputs input data and motion data generated by the motion generation unit 30 to the model E consisting of a neural network, and outputs an identification feature extraction value (step S5). Specifically, with respect to the input data x₁ₐ and the motion data f_{1c} output by the individual feature conversion unit 31, the individual identification feature extraction unit 41 output an individual identification feature extraction value {e^{r}ₐ, e^{f}_{c}}. Moreover, with respect to the input data x₁ₐ and the motion data g₂ₐ output by the motion feature conversion unit 32, the motion identification feature extraction unit 42 outputs a motion identification feature extraction value {d^{r}₁, d^{f}₂}.

(8) Calculate Loss Function

The loss function calculation unit 51 calculates, with respect to the input data and data {x₁ₐ, f_{1c}, g₂ₐ} generated by the individual feature conversion unit 31 and the motion feature conversion unit 32, a loss function for identifying whether they are generated data or input data. The loss function calculation unit 51 calculates the difference by using the adversarial learning loss function from the individual identification feature extraction value {e^{r}ₐ, e^{f}_{c}} and the motion identification feature extraction value {d^{r}₁, d^{f}₂} (step S6).

Further, the loss function calculation unit 51 calculates a loss function for classification with resect to the feature vector bi a extracted by the basic feature extraction unit 22, the feature vector m_{2b} extracted by the motion feature extraction unit 23, and the feature vector p_{3c} extracted by the individual feature extraction unit 21. For example, the loss function calculation unit 51 uses a cross entropy function to calculate the difference from each corresponding correct label {x, 2, c} (step S6).

Further, the loss function calculation unit 51 calculates a loss function for allowing the difference between the motion data {f₁ₐ, g₁ₐ}, cyclically generated by the individual feature conversion unit 31 and the motion feature conversion unit 32, and the input data x₁ₐ to be smaller. For example, the loss function calculation unit 51 calculates the difference by using a mean absolute error function, a mean squared error function, or the like (step S6).

(9) Update Learning/Model Parameter

The learning unit 52 performs learning by using an optimization method of a neural network based on the value calculated by the loss function calculation unit 51, and updates the weight of each of the models B M, P, F, G, E, and D consisting of a neural network. That is, the learning unit learns a conversion model constituting each unit, and updates the parameter of each model (step S7).

Repeat (2)-(9)

The processing described above is repeated until the models each consisting of a neural network is sufficiently learned. The end of repetition is determined by previously setting the number of times of repetition or setting a threshold for the value of a loss function.

(10) Replace Motion Identification

When the number of times of repetition becomes equal to or larger than a given value, motion identification for identifying the motion data g₂ₐ output by the motion feature conversion unit 32 is replaced with individual identification at a certain rate. That is, with respect to the input data x₁ₐ and the motion data g₂ₐ output by the motion feature conversion unit 32, the individual identification feature extraction unit 41 outputs an individual identification feature extraction value {e^{r}ₐ, e^{f}a}. Then, learning as described above is performed by using the individual identification feature extraction value, and the parameter of each model is updated. Thereby, the model G consisting of a neural network is learned such that the motion data g₂ₐ output by the motion feature conversion unit 32 has not only a motion style 2 but also an individual feature “a”. That is, from the motion data generated by the motion feature conversion unit 32, an individual identification feature is extracted by the individual identification feature extraction unit 41, and learning is performed in such a manner that the individual feature will not be changed by the motion feature conversion unit 32. Note that the number of time of repetition for starting replacement and the rate of replacement may be previously given or set, or determined according to the value of a loss function.

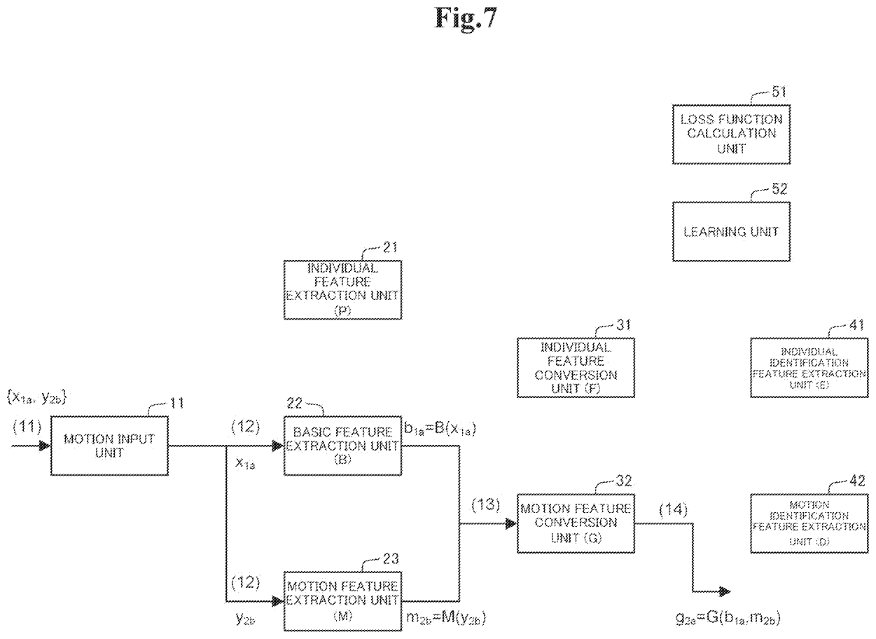

Next, an inference phase will be described with reference to FIG. 7.

(11) Read Motion Data

First, the motion input unit 11 reads motion data to be converted. Here, it is assumed that the motion input unit 11 reads motion data {x₁ₐ, y_{2b}}, and desires to convert the motion data x₁ₐ, that is, a motion style 1 of the individual feature “a”, into the motion style 2.

(12) Input Motion

The motion input unit 11 inputs the motion data x₁ₐ to the basic feature extraction unit 22, and inputs the motion data y_{2b} to the motion feature extraction unit 23.

(13) Extract Feature Value

The basic feature extraction unit 22 inputs the motion data x₁ₐ to the model B and converts it into the basic feature vector b₁ₐ. Moreover, the motion feature extraction unit 23 inputs the motion data y_{2b} to the model M and converts it into the motion feature vector m_{2b}.

(14) Generate Motion

The motion feature conversion unit 32 generates the motion data g₂ₐ in which the motion style is converted, based on the motion feature vector m_{2b} and the basic feature vector b₁ₐ. At that time, since the model G is learned to have the individual feature “a” as described above, motion data in which the motion feature 1 is converted to the motion feature 2 is generated from the motion data x₁ₐ, while the individual feature “a” is not changed. As described above, according to the information processing device 1 of the present disclosure, it is possible to generate motion data in various situations of a person while reflecting the individual feature of the person. As a result, it is possible to predict a change in the motion of a person with high accuracy. In addition, when generating such motion data, learning can be performed without a need of pieces of motion data forming a pair before and after the motion for the same person. As a result, a change in the motion of a person can be predicted with higher accuracy at low cost.

[Modification]

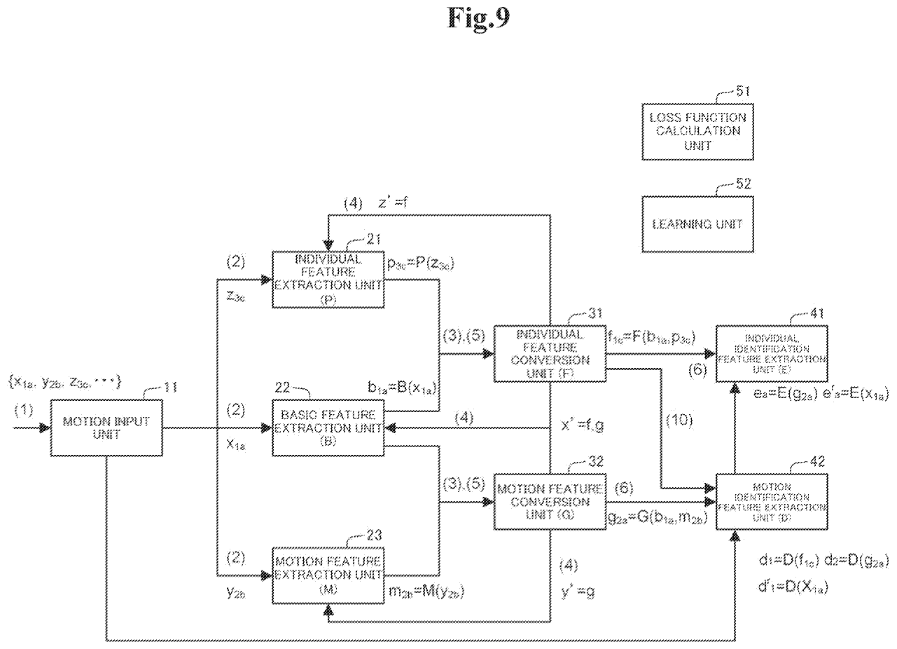

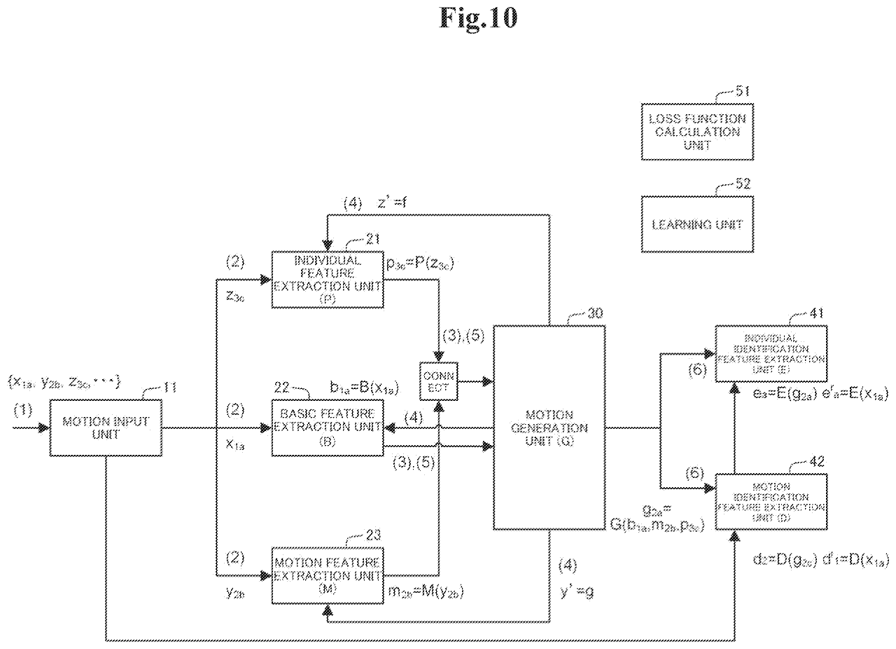

Next, a modification of the configuration and operation of the information processing device 1 described above will be described with reference to FIGS. 9 and 10. First, in the learning phase described above, at (10), when the number of times of repetition of learning becomes equal to or larger than a given number, the information processing device 1 replaces motion identification for identifying the motion data g₂ₐ output by the motion feature conversion unit 32 with individual identification at a certain rate. Instead, in the present modification, as illustrated in FIG. 9, when the number of times of repetition of learning becomes equal to or larger than a given number, the information processing device 1 replaces the individual identification that identifies the motion data f_{1c} output by the individual feature conversion unit 31 with the motion identification at a certain rate. That is, with respect to the input data x₁ₐ and the motion data f_{1c} output by the individual feature conversion unit 31, the motion identification feature extraction unit 42 outputs a motion identification feature extraction value. Then, the learning described above is performed using the motion identification feature extraction value. Therefore, learning is performed in such a manner that the motion style will not be changed in the individual feature conversion unit 31. As a result, from the motion data x₁ₐ for example, it is possible to generate motion data in which the individual feature “a” is converted to the individual feature “b”, while the motion style 1 is not changed.

Further, while the case where the motion generation unit 30 is configured of the individual feature conversion unit 31 and the motion feature conversion unit 32 has been described, the motion generation unit 30 may not be divided into the two units. For example, as illustrated in FIG. 10, the motion generation unit 30 may be configured of the model G consisting of one neural network. In that case, an individual feature vector output by the individual feature extraction unit 21 and a motion feature vector output by the motion feature extraction unit 23 may be connected and input to the model G, whereby the motion data can be output in the same manner as the above description.

Moreover, the basic feature extraction unit 22 may be configured to be divided for the individual feature extraction unit 21 and for the motion feature extraction unit 23, although not illustrated. This means that two basic feature extraction units 22 may be prepared and divided into a unit that forms a set with the individual feature extraction unit 21 and a unit that forms a set with the motion feature extraction unit 23, so that the system for processing the individual feature and the system for processing the motion style may be separated.

### Second Example Embodiment

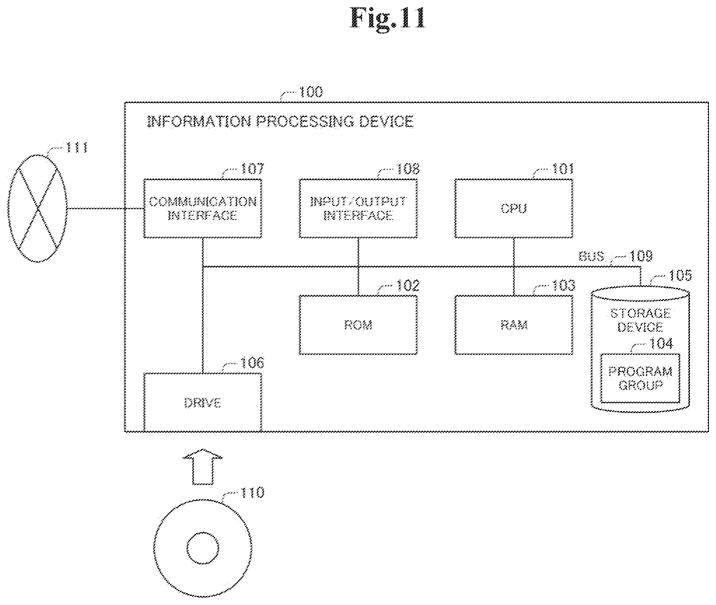

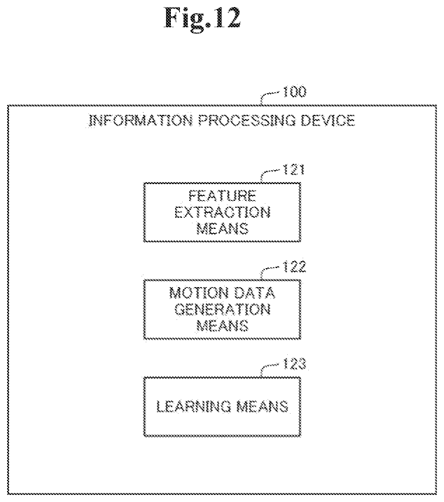

Next, a second example embodiment of the present disclosure will be described with reference to FIGS. 11 and 12. FIGS. 11 and 12 are block diagrams illustrating a configuration of an information processing device according to the second example embodiment. Note that the present embodiment shows the outline of the configuration of the information processing device described in the embodiment described above.

First, a hardware configuration of an information processing device 100 in the present embodiment will be described with reference to FIG. 11. The information processing device 100 is configured of a typical information processing device, having a hardware configuration as described below as an example.
Central Processing Unit (CPU) 101 (arithmetic device)
Read Only Memory (ROM) 102 (storage device)
Random Access Memory (RAM) 103 (storage device)
Program group 104 to be loaded to the RAM 103
Storage device 105 storing therein the program group 104
Drive 106 that performs reading and writing on a storage medium 110 outside the information processing device
Communication interface 107 connecting to a communication network 111 outside the information processing device
Input/output interface 108 for performing input/output of data
Bus 109 connecting the respective constituent elements

Note that FIG. 11 illustrates an example of the hardware configuration of an information processing device that is the information processing device 100. The hardware configuration of the information processing device is not limited to that described above. For example, the information processing device may be configured of part of the configuration described above, such as without the drive 106. Moreover, instead of the CPU, the information processing device may use a Graphic Processing Unit (GPU), a Digital Signal Processor (DSP), a Micro Processing Unit (MPU), a Floating Point number processing Unit (FPU), a Physics Processing Unit (PPU), a Tensor Processing Unit (TPU), a quantum processor, a microcontroller, or a combination thereof.

The information processing device 100 can construct, and can be equipped with, a feature extraction means 121, a motion data generation means 122, and a learning means 123 illustrated in FIG. 12, through acquisition and execution of the program group 104 by the CPU 101. Note that the program group 104 is stored in the storage device 105 or the ROM 102 in advance, and is loaded to the RAM 103 and executed by the CPU 101 as needed. Further, the program group 104 may be provided to the CPU 101 via the communication network 111, or may be stored on the storage medium 110 in advance and read out by the drive 106 and supplied to the CPU 101. However, the feature extraction means 121, the motion data generation means 122, and the learning means 123 may be constructed by dedicated electronic circuits for implementing such means.

The feature extraction means 121 extracts, from input data that is motion data representing a motion of a person, basic feature data representing a feature of motion data corresponding to the basic motion set with respect to the motion, motion feature data representing a feature of motion data corresponding to a motion style set with respect to the motion, and person feature data representing a feature of motion data corresponding to the person.

The motion data generation means 122 generates first motion data on the basis of the basic feature data and the motion feature data, and generates second motion data on the basis of the basic feature data and the person feature data.

The learning means 123 learns the feature extraction means and the motion data generation means on the basis of the first motion data and the second motion data.

Since the present disclosure is configured as described above, when generating motion data in which the motion style has been changed from the basic motion, it is possible to reflect the characteristics of a person. As a result, it is possible to predict a change in the motion of a person with high accuracy.

Note that the program described above can be supplied to a computer by being stored in a non-transitory computer readable medium of any type. Non-transitory computer-readable media include tangible storage media of various types. Examples of non-transitory computer-readable media include magnetic storage media (for example, flexible disk, magnetic tape, and hard disk drive), magneto-optical storage media (for example, magneto-optical disk), a CD-ROM (Read Only Memory), a CD-R, a CD-R/W, and semiconductor memories (for example, mask ROM, PROM (Programmable ROM), EPROM (Erasable PROM), flash ROM, and RAM (Random Access Memory)). The program may be supplied to a computer by a transitory computer-readable medium of any type. Examples of transitory computer-readable media include electric signals, optical signals, and electromagnetic waves. A transitory computer readable medium can supply a program to a computer via a wired communication channel such as a wire or an optical fiber, or a wireless communication channel.

While the present disclosure has been described with reference to the example embodiments described above, the present disclosure is not limited to the above-described embodiments. The form and details of the present disclosure can be changed within the scope of the present disclosure in various manners that can be understood by those skilled in the art. Further, at least one of the functions of the feature extraction means 121, the motion data generation means 122, and the learning means 123 described above may be carried out by an information processing device provided and connected to any location on the network, that is, may be carried out by so-called cloud computing.

### <Supplementary Notes>

The whole or part of the example embodiments disclosed above can be described as the following supplementary notes. Hereinafter, outlines of the configurations of an information processing device, an information processing method, and a program, according to the present disclosure, will be described. However, the present disclosure is not limited to the configurations described below.

### (Supplementary Note 1)

An information processing device comprising:
a feature extraction means for extracting, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
a motion data generation means for generating first motion data on a basis of the basic feature data and the motion feature data, and generating second motion data on a basis of the basic feature data and the person feature data; and
a learning means for learning the feature extraction means and the motion data generation means on a basis of the first motion data and the second motion data.

### (Supplementary Note 2)

The information processing device according to supplementary note 1, wherein in the feature extraction means and the motion data generation means, the learning means learns the feature extraction means and the motion data generation means so as to generate the first motion data and the second motion data from the input data and generate the input data from each of the first motion data and the second motion data.

### (Supplementary Note 3)

The information processing device according to supplementary note 1, further comprising
an identification feature extraction means for generating an identification feature value that is a feature value for identifying whether each of the first motion data and the second motion data is data generated by the motion data generation means or the input data, wherein
the learning means learns the feature extraction means, the motion data generation means, and the identification feature extraction means by using the identification feature value.

### (Supplementary Note 4)

The information processing device according to supplementary note 3, wherein
the identification feature extraction means includes a motion identification feature extraction means for generating the identification feature value corresponding to each of the input data and the first motion data, and an individual identification feature extraction means for generating the identification feature value corresponding to each of the input data and the second motion data, and
the learning means learns the feature extraction means, the motion data generation means, and the identification feature extraction means by performing adversarial learning with use of the identification feature value generated by the motion identification feature extraction means and the identification feature value generated by the individual identification feature extraction means.

### (Supplementary Note 5)

The information processing device according to supplementary note 4, wherein
the individual identification feature means further generates the identification feature value corresponding to the first motion data, and
the learning means learns the feature extraction means, the motion data generation means, and the identification feature extraction means by performing adversarial learning with use of the identification feature value corresponding to each of the input data and the second motion data and the identification feature value corresponding to each of the input data and the first motion data, generated by the individual identification feature extraction means.

### (Supplementary Note 6)

The information processing device according to supplementary note 4, wherein
the motion identification feature extraction means further generates the identification feature value corresponding to the second motion data, and
the learning means learns the feature extraction means, the motion data generation means, and the identification feature extraction means by performing adversarial learning with use of the identification feature value corresponding to each of the input data and the first motion data and the identification feature value corresponding to each of the input data and the second motion data, generated by the motion identification feature extraction means.

### (Supplementary Note 7)

The information processing device according to supplementary note 1, wherein
the learning means learns the feature extraction means and the motion data generation means in such a manner that the basic feature data, the motion feature data, and the person feature data are classified into predetermined labels respectively.

### (Supplementary Note 8)

An information processing method comprising:
by a feature extraction means, extracting, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation means, generating first motion data on a basis of the basic feature data and the motion feature data, and generating second motion data on a basis of the basic feature data and the person feature data; and
by a learning means, learning the feature extraction means and the motion data generation means on a basis of the first motion data and the second motion data.

### (Supplementary Note 9)

The information processing method according to supplementary note 8, further comprising
by an identification feature extraction means, generating an identification feature value that is a feature value for identifying whether each of the first motion data and the second motion data is data generated by the motion data generation means or the input data; and
by the learning means, learning the feature extraction means, the motion data generation means, and the identification feature extraction means by using the identification feature value.

### (Supplementary Note 10)

A program for causing a computer to execute processing to:
by a feature extraction means, extract, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation means, generate first motion data on a basis of the basic feature data and the motion feature data, and generate second motion data on a basis of the basic feature data and the person feature data; and
by a learning means, learn the feature extraction means and the motion data generation means on a basis of the first motion data and the second motion data.

### REFERENCE SIGNS LIST

1 information processing device
11 motion input unit
20 feature extraction unit
21 individual feature extraction unit
22 basic feature extraction unit
23 motion feature extraction unit
30 motion generation unit
31 individual feature conversion unit
32 motion feature conversion unit
40 identification feature extraction unit
41 individual identification feature extraction unit
42 motion identification feature extraction unit
51 loss function calculation unit
52 learning unit
100 information processing device
101 CPU
102 ROM
103 RAM
104 program group
105 storage device
106 drive
107 communication interface
108 input/output interface
109 bus
110 storage medium
111 communication network
121 feature extraction means
122 motion data generation means
123 learning means

1. An information processing device comprising:
at least one memory configured to store instructions; and
at least one processor configured to execute instructions to:
by a feature extraction unit, extract, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation unit, generate first motion data on a basis of the basic feature data and the motion feature data, and generate second motion data on a basis of the basic feature data and the person feature data; and
learn the feature extraction unit and the motion data generation unit on a basis of the first motion data and the second motion data.

2. The information processing device according to claim 1, wherein the at least one processor is configured to execute the instructions to
in the feature extraction unit and the motion data generation unit, learn the feature extraction unit and the motion data generation unit so as to generate the first motion data and the second motion data from the input data and generate the input data from each of the first motion data and the second motion data.

3. The information processing device according to claim 1, wherein the at least one processor is configured to execute the instructions to
by an identification feature extraction unit, generate an identification feature value that is a feature value for identifying whether each of the first motion data and the second motion data is data generated by the motion data generation unit or the input data; and
learn the feature extraction unit, the motion data generation unit, and the identification feature extraction unit by using the identification feature value.

4. The information processing device according to claim 3, wherein
the identification feature extraction unit includes a motion identification feature extraction unit that generates the identification feature value corresponding to each of the input data and the first motion data, and an individual identification feature extraction unit that generates the identification feature value corresponding to each of the input data and the second motion data, and
the at least one processor is configured to execute the instructions to learn the feature extraction unit, the motion data generation unit, and the identification feature extraction unit by performing adversarial learning with use of the identification feature value generated by the motion identification feature extraction unit and the identification feature value generated by the individual identification feature extraction unit.

5. The information processing device according to claim 4, wherein
the individual identification feature unit further generates the identification feature value corresponding to the first motion data, and
the at least one processor is configured to execute the instructions to learn the feature extraction unit, the motion data generation unit, and the identification feature extraction unit by performing adversarial learning with use of the identification feature value corresponding to each of the input data and the second motion data and the identification feature value corresponding to each of the input data and the first motion data, generated by the individual identification feature extraction unit.

6. The information processing device according to claim 4, wherein
the motion identification feature extraction unit further generates the identification feature value corresponding to the second motion data, and
the at least one processor is configured to execute the instructions to learn the feature extraction unit, the motion data generation unit, and the identification feature extraction unit by performing adversarial learning with use of the identification feature value corresponding to each of the input data and the first motion data and the identification feature value corresponding to each of the input data and the second motion data, generated by the motion identification feature extraction unit.

7. The information processing device according to claim 1, wherein the at least one processor is configured to execute the instructions to
learn the feature extraction unit and the motion data generation unit in such a manner that the basic feature data, the motion feature data, and the person feature data are classified into predetermined labels respectively.

8. An information processing method comprising:
by a feature extraction unit, extracting, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation unit, generating first motion data on a basis of the basic feature data and the motion feature data, and generating second motion data on a basis of the basic feature data and the person feature data; and
by a learning unit, learning the feature extraction unit and the motion data generation unit on a basis of the first motion data and the second motion data.

9. The information processing method according to claim 8, further comprising
by an identification feature extraction unit, generating an identification feature value that is a feature value for identifying whether each of the first motion data and the second motion data is data generated by the motion data generation unit or the input data; and
by the learning unit, learning the feature extraction unit, the motion data generation unit, and the identification feature extraction unit by using the identification feature value.

10. A non-transitory computer-readable medium storing thereon a program comprising instructions for causing a computer to execute processing to:
by a feature extraction unit, extract, from input data that is motion data representing a motion of a person, basic feature data representing a feature of the motion data corresponding to a basic motion set with respect to the motion, motion feature data representing a feature of the motion data corresponding to a motion style set with respect to the motion, and person feature data representing a feature of the motion data corresponding to the person;
by a motion data generation unit, generate first motion data on a basis of the basic feature data and the motion feature data, and generate second motion data on a basis of the basic feature data and the person feature data; and
by a learning unit, learn the feature extraction unit and the motion data generation unit on a basis of the first motion data and the second motion data.
